(19) United States
(12) Patent Application Publication
(10) Pub. No.: US 2014/0010776 A1
(43) Pub. Date: Jan. 9, 2014

(54) POLYURETHANE UREA MIXTURE FOR HAIR COSMETICS

(71) Applicants: Sophie Viala, Koln (DE); Sebastian Dörr, Dusseldorf (DE)

(72) Inventors: Sophie Viala, Koln (DE); Sebastian Dörr, Dusseldorf (DE)

(73) Assignee: BAYER INTELLECTUAL PROPERTY GMBH, Monheim (DE)

(21) Appl. No.: 14/006,018

(22) PCT Filed: Mar. 21, 2012
(86) PCT No.: PCT/EP2012/054976

§ 371 Date: Sep. 20, 2013

(30) Foreign Application Priority Data
Mar. 25, 2011 (EP) 11159697.9

(51) Int. Cl. A61K 8/87; A61Q 5/06

(52) U.S. Cl. CPC A61K 8/87; A61Q 5/06
USPC 424/70.11; 525/440.01

(57) ABSTRACT

The invention relates to a specific polyurethane urea, which can be obtained by reacting a single polyisocyanate component, at least one polymeric polyol component, at least one hydrophilizing component and a single amino-functional chain extender component, wherein the polyisocyanate component comprises more than 75 mol % isophorone diisocyanate and the amino-functional chain extender component comprises more than 75 mol % isophorone diamine. Said polyurethane urea can be used in the field of hair cosmetics, in particular in the form of a mixture with conventional solvents. The invention further relates to the use of the polyurethane urea in cosmetics, to a cosmetic composition containing the polyurethane urea, to the use of the cosmetic composition in cosmetics, and to a method for forming hair styles using the cosmetic composition.

The present invention relates to a specific polyurethane urea which can be used in particular in the form of a mixture with customary solvents in the field of hair cosmetics. Further subjects of the invention are the use of the polyurethane urea in cosmetics, a cosmetic composition comprising the polyurethane urea, the use of the cosmetic composition in cosmetics and a method for forming hairstyles using the cosmetic composition.

Diverse hairstyles are created and stabilized using products which are known as hair-setting compositions. Hair-setting compositions come in most cases in the form of mousse setting compositions or hairsprays, differing little in their composition. Mousse setting compositions are applied to wet hair as aids for modeling the hairstyle. In contrast to this, hairsprays are applied to dry ready-styled hair to fix the hairstyle.

In the case of hairsprays and mousse setting compositions, the compositions for fixing or creating the hairstyle are usually in the form of preparations that can be sprayed from aerosol containers, squeezy bottles or by a pump, spraying or foaming devices, said preparations consisting of an alcoholic or aqueous-alcoholic solution of film-forming natural or synthetic polymers. These polymers can be selected from the group of nonionic, cationic, amphoteric or anionic polymers.

The film-forming polymers used in the prior art are often anionic or amphoteric polymers based on acrylates. However, the use of polyurethanes and polyurethane ureas as film formers is also known. Thus, hair setting compositions are described for example in WO 2009/118105 A1 which obtain a polyurethane urea which is obtainable by reacting a water-insoluble non-water-dispersible isocyanate-functional polyurethane prepolymer with an amino-functional compound. The hair setting compositions disclosed herein are highly suited for stabilizing hairstyles. However, transparent mixtures cannot be produced from the known water-based polyurethane ureas. For this reason, systems which comprise the solvents customary in the field of hair cosmetics, such as water and ethanol, are cloudy. This is perceived as being disadvantageous for many applications.

It was therefore an object of the present invention to provide a polyurethane urea which has the advantageous fixing properties of the systems known from WO 2009/118105 A1 and from which, moreover, can be produced clear mixtures in solvents customary in the field of cosmetics.

This object is achieved according to the invention by a polyurethane urea which is obtainable by reacting

a) a single polyisocyanate component,

b) at least one polymeric polyol component,

c) at least one hydrophilizing component and

d) a single amino-functional chain extender component,

where the polyisocyanate component a) comprises >75 mol % isophorone diisocyanate (IPDI) and the amino-functional chain extender component c) comprises >75 mol % isophoronediamine (IPDA).

According to the invention, an amino-functional chain extender component is understood as meaning a component which comprises at least one compound with two isocyanate-reactive amino groups and no hydrophilizing groups.

According to a first preferred embodiment of the polyurethane urea according to the invention, the polyisocyanate component a) can comprise 80 mol %, preferably 85 mol %, further preferably 95 mol % and particularly preferably 100 mol %, IPDI.

Further polyisocyanates of component a) that can be used in addition to IPDI in a molar fraction of less than 25 mol %, are the aromatic, araliphatic, aliphatic or cycloaliphatic polyisocyanates with an NCO functionality of 2 that are known per se to the person skilled in the art.

Examples of such polyisocyanates are 1,4-butylene diisocyanate, 1,6-hexamethylene diisocyanate (HDI), 2,2,4 and/or 2,4,4-trimethylhexamethylene diisocyanate, the isomeric bis(4,4′-isocyanatocyclohexyl)methanes or their mixtures of any desired isomer content, 1,4-cyclohexylene diisocyanate, 1,4-phenylene diisocyanate, 2,4- and/or 2,6-tolylene diisocyanate, 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI), alkyl 2,6-diisocyanatohexanoate (lysine diisocyanates) with C1-C8-alkyl groups, and 4-isocyanatomethyl 1,8-octanediisocyanate (nonane triisocyanate) and triphenylmethane 4,4′,4″-triisocyanate.

As well as the aforementioned polyisocyanates, it is also possible to co-use in part modified diisocyanates or triisocyanates with a uretdione, isocyanurate, urethane, allophanate, biuret, iminooxadiazinedione and/or oxadiazinetrione structure.

They are preferably polyisocyanates or polyisocyanate mixtures of the type specified above with exclusively aliphatically and/or cycloaliphatically bonded isocyanate groups and an average NCO functionality of the mixture of 2 to 4, preferably of 2 to 2.6 and particularly preferably of 2 to 2.4.

If further polyisocyanates are used in component a) besides IPDI, particular preference is given to using 1,6-hexamethylenediisocyanate, the isomeric bis(4,4′-isocyanatocyclohexyl)methanes, and mixtures thereof.

It is likewise preferred if the polymeric polyol component b) has a number-average molecular weights of ≧400 and ≦8000 g/mol, particularly preferably from 600 to 3000 g/mol and/or an average OH functionalities of 1.5 to 6, preferably from 1.8 to 3 and particularly preferably from 1.9 to 2.1.

It is likewise advantageous if the polymeric polyol component b) comprises a polyester, preferably a polyester based on adipic acid, or consists thereof.

Possible constituents of the polymeric polyol component b) are the polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols known per se in polyurethane coating technology. These can be used in b) individually or in any desired mixtures with one another.

Polyester polyols are for example the polycondensates, known per se, of di- and optionally tri-, and tetraols and di- and optionally tri- and tetracarboxylic acids or hydroxycarboxylic acids or lactones. Instead of the free polycarboxylic acids, the corresponding polycarboxylic anhydrides or corresponding polycarboxylic esters of lower alcohols can also be used for producing the polyesters.

Examples of diols suitable for this are ethylene glycol, butylene glycol, diethylene glycol, triethylene glycol, polyalkylene glycols, such as polyethylene glycol, also 1,2-propanediol, 1,3-propanediol, butanediol(1,3), butanediol(1,4), hexanediol(1,6) and isomers, neopentyl glycol or neopentyl glycol hydroxypivalate, with hexanediol(1,6) and isomers, neopentyl glycol and neopentyl glycol hydroxypivalate being preferred. In addition, polyols such as trimethylolpropane, glycerol, erythritol, pentaerythritol, trimethylolbenzene or trishydroxyethyl isocyanurate can also be used.

Dicarboxylic acids which can be used are phthalic acid, isophthalic acid, terephthalic acid, tetra-hydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. The corresponding anhydrides can also be used as acid source.

If the average functionality of the polyol to be esterified is greater than 2, it is also possible to additionally co-use monocarboxylic acids, such as benzoic acid and hexanecarboxylic acid.

Preferred acids are aliphatic or aromatic acids of the aforementioned type. Particular preference is given to adipic acid, isophthalic acid and optionally trimellitic acid, very particularly preferably adipic acid.

Hydroxycarboxylic acids which can be co-used as reactants in the production of a polyester polyol with terminal hydroxyl groups are for example hydroxycaproic acid, hydroxybutyric acid, hydroxy-decanoic acid, hydroxystearic acid and the like. Suitable lactones are caprolactone, butyrolactone and homologs. Preference is given to caprolactone.

In component b), it is also possible to use polycarbonates having hydroxyl groups, preferably polycarbonate diols, having number-average molecular weights Mn of 400 to 8000 g/mol, preferably from 600 to 3000 g/mol. These are obtainable by reaction of carbonic acid derivatives, such as diphenyl carbonate, dimethyl carbonate or phosgene, with polyols, preferably diols.

Examples of such diols are ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentanediol-1,3, dipropylene glycol, polypropylene glycols, dibutylene glycol, polybutylene glycols, bisphenol A and lactone-modified diols of the aforementioned type. The polycarbonates having hydroxyl groups are preferably linear in structure.

Polyether polyols can likewise be used in component b). Of suitability are for example the polytetramethylene glycol polyethers known per se in polyurethane chemistry, as are obtainable by polymerization of tetrahydrofuran by means of cationic ring opening.

Likewise suitable polyether polyols are the addition products, known per se, of styrene oxide, ethylene oxide, propylene oxide, butylene oxides and/or epichlorohydrin onto di- or polyfunctional starter molecules.

Suitable starter molecules that can be used are all compounds known according to the prior art, such as, for example, water, butyl diglycol, glycerol, diethylene glycol, trimethylolpropane, propylene glycol, sorbitol, ethylenediamine, triethanolamine, 1,4-butanediol. Preferred starter molecules are water, ethylene glycol, propylene glycol, 1,4-butanediol, diethylene glycol and butyl diglycol.

According to a further advantageous embodiment of the polyurethane urea according to the invention, it is provided that the hydrophilizing component c) is an anionically hydrophilizing component and preferably a sulfonate.

Suitable anionically or potentially anionically hydrophilizing compounds of component c) are compounds which have at least one isocyanate-reactive group such as a hydroxyl group or amino group and at least one functionality such as e.g. —COO-M⁺, —SO₃M⁺, —PO(O-M⁺)₂ where M⁺ is for example metal cation, H⁺, NH₄⁺, NHR₃⁺, where R can in each case be a C1-C12-alkyl radical, C5-C6-cycloalkyl radical and/or a C2-C4-hydroxyalkyl radical, which enters into a pH-dependent dissociation equilibrium upon interaction with aqueous media and, in so doing, can be negatively or neutrally charged. Suitable anionically or potentially anionically hydrophilizing compounds are mono- and dihydroxycarboxylic acids, mono- and dihydroxysulfonic acids, and also mono- and dihydroxyphosphonic acids and their salts. Examples of such anionic or potentially anionic hydrophilizing agents are dimethylolpropionic acid, dimethylolbutyric acid, hydroxypivalic acid, malic acid, citric acid, glycolic acid, lactic acid and the propoxylated adduct of 2-butenediol and NaHSO₃, as is described in DE-A 2 446 440, pages 5-9, formula I-III. Preferred anionic or potentially anionic hydrophilizing agents of component c) are those of the aforementioned type which have carboxylate or carboxylic acid groups and/or sulfonate groups.

Suitable nonionically hydrophilizing compounds of component c) are e.g. polyoxyalkylene ethers which contain at least one hydroxy or amino group, preferably at least one hydroxy group.

Examples are the monohydroxy-functional polyalkylene oxide polyether alcohols having on statistical average 5 to 70, preferably 7 to 55, ethylene oxide units per molecule, as are accessible in a manner known per se by alkoxylation of suitable starter molecules (described e.g. in Ullmanns Encyclopädie der technischen Chemie [Ullmann's Encyclopedia of Industrial Chemistry], 4th edition, volume 19, Verlag Chemie, Weinheim pp. 31-38). These compounds are either pure polyethylene oxide ethers or mixed polyalkylene oxide ethers, in which case, however, they then contain at least 30 mol %, preferably at least 40 mol %, based on all contained alkylene oxide units, of ethylene oxide units. Particularly preferred nonionic compounds are monofunctional, mixed polyalkylene oxide polyethers which have 40 to 100 mol % ethylene oxide units and 0 to 60 mol % propylene oxide units.

For the hydrophilization it is also possible to use mixtures of anionic or potentially anionic hydrophilizing agents and nonionic hydrophilizing agents.

In an embodiment of the invention, it is provided that the amino-functional chain extender component d) can comprise 85 mol %, preferably 95 mol % and particularly preferably 100 mol %, IPDA.

As further constituents of the amino-functional chain extender d), further NH₂— and/or NH— functional compounds can be used besides IPDI.

Preferably, the chain extension/termination is carried out before dispersion in water, the isocyanate groups reacting with the chain extender to give urea groups.

Suitable components—which can be used in addition to IPDA in a molar fraction of less than 25%—are di- or polyamines such as 1,2-ethylenediamine, 1,2- and 1,3-diaminopropane, 1,4-diamino-butane, 1,6-diaminohexane, 2,2,4- and 2,4,4-trimethylhexamethylenediamine, 2-methylpenta-methylenediamine, diethylenetriamine, triaminononane, 1,3- and 1,4-xylylenediamine, α,α,α′,α′-tetramethyl-1,3- and -1,4-xylylenediamine and 4,4-diaminodicyclohexylmethane and/or dimethylethylenediamine. Hydrazine or and hydrazides such as adipic dihydrazide are likewise possible.

Apart from components a) to d), further building blocks can also be used for producing the polyurethane urea according to the invention.

Examples are hydroxy-functional compounds with molecular weights of 62 to 399 g/mol, such as, for example, polyols of the specific molecular weight range having up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A, (2,2-bis(4-hydroxycyclohexyl)propane), trimethylolpropane, glycerol, pentaerythritol. Furthermore, monofunctional isocyanate-reactive amine compounds can be used, such as, for example, methylamine, ethylamine, propylamine, butylamine, octylamine, laurylamine, stearylamine, isononyloxypropylamine, dimethylamine, diethylamine, dipropylamine, dibutylamine, n-methylaminopropylamine, diethyl(methyl)-aminopropylamine, morpholine, piperidine.

Apart from components a) to d), no further building blocks are preferably used for producing the polyurethane urea according to the invention.

The preparation of the polyurethane urea according to the invention can take place by processes known to the person skilled in the art in one or more stage(s) in homogeneous or for multistage reaction and be carried out in part in disperse phase. A dispersing, emulsifying or dissolution step preferably takes place following completely or partially carried out polyaddition from a) to d). Subsequently, a further polyaddition or modification optionally takes place in disperse or dissolved (homogeneous) phase. In this connection, it is possible to use all processes known from the prior art, such as e.g. prepolymer mixing processes, acetone processes or melt dispersion processes. Preference is given to working in accordance with the acetone process.

A further subject of the present invention is the use of a polyurethane urea according to the invention in cosmetics, preferably in the field of hair cosmetics, particularly preferably in the field of hairstyling.

A yet further subject of the present invention is a cosmetic composition comprising a solvent mixture and a polyurethane urea according to the invention, where the solvent mixture comprises ethanol and water.

The solvent mixture can optionally comprise further cosmetically suitable solvents. Preferred solvents are aliphatic alcohols with C2-4 carbon atoms such as isopropanol, t-butanol, n-butanol; polyols such as propylene glycol, glycerol, ethylene glycol and polyol ether; acetone; unbranched or branched hydrocarbons such as pentane, hexane, isopentane and cyclic hydrocarbons such as cyclopentane and cyclohexane; and mixtures thereof.

The solvent mixture can comprise ≧10% by weight and ≦98% by weight, preferably ≧15% by weight and ≦98% by weight, further preferably ≧20% by weight and ≦90% by weight and particularly preferably ≧20% by weight and ≦80% by weight, ethanol.

It is likewise possible that the solvent mixture consists of water and ethanol.

According to one embodiment of the composition according to the invention, it is provided that it comprises ≧0.1% by weight and ≦30% by weight, preferably ≧0.1% by weight and ≦20% by weight, further preferably ≧0.5% by weight and ≦15% by weight and particularly preferably ≧0.5% by weight and ≦10% by weight, of the polyurethane urea.

Preference is also given to a composition which comprises ≧10% by weight and ≦98% by weight, preferably ≧20% by weight and ≦98% by weight, further preferably ≧30% by weight and ≦98% by weight and particularly preferably ≧40% by weight and ≦98% by weight, of the solvent mixture.

The turbidity values of the systems are measured using a turbidity meter model 2100 P from Hach (turbidity measurement from 0.01 to 1000 NTU). Before the measurement, the instrument was calibrated with formazine standards (standard 1/20 NTU, standard 2/100 NTU and standard 3/800 NTU). The lowest possible values are desired since these indicate low turbidity.

A subject of the invention is also the use of a cosmetic composition according to the invention in cosmetics, preferably in the field of hair cosmetics, particularly preferably in the field of hairstyling.

A yet further subject of the present invention is a method for shaping hairstyles in which a cosmetic composition according to the invention is applied to hair.

Besides the polyurethane described above, the composition according to the invention can comprise further suitable film formers, which can in particular also contribute to the setting and the styling of the hair.

The fraction of one or more further film formers can be from 0 to 20% by weight and in particular 0 to 10% by weight, based on the total formulation.

The further film former or formers are advantageously selected from the group of nonionic, anionic, amphoteric and/or cationic polymers and mixtures thereof.

Suitable nonionic polymers, which can be present in the composition according to the invention on their own or in mixtures, preferably also with anionic and/or amphoteric and/or zwitterionic polymers, are selected from the group:
polyalkyloxazolines;
vinyl acetate homo- or copolymers, these including for example copolymers of vinyl acetate and acrylic esters, copolymers of vinyl acetate and ethylene, copolymers of vinyl acetate and maleic esters;
acrylic ester copolymers such as e.g. the copolymers of alkyl acrylate and alkyl methacrylate, copolymers of alkyl acrylate and urethanes;
copolymers of acrylonitrile and nonionic monomer selected from butadiene and (meth)acrylate;
styrene homo- and copolymers, these including for example homopolystyrene, copolymers of styrene and alkyl(meth)acrylate, copolymers of styrene, alkyl methacrylate and alkyl acrylate, copolymers of styrene and butadiene, copolymers of styrene, butadiene and vinylpyridine;
polyamides;
vinyl lactam homo- or copolymers, such as vinylpyrrolidone homo- or copolymers; these including for example polyvinylpyrrolidone, polyvinylcaprolactam, copolymers of N-vinylpyrrolidone and vinyl acetate and/or vinyl propionate in various concentration ratios, polyvinylcaprolactam, polyvinylamides and salts thereof, and also copolymers of vinylpyrrolidone and dimethylaminoethyl methacrylate, terpolymers of vinylcaprolactam, vinylpyrrolidone and dimethylaminoethyl methacrylate;
polysiloxanes; and
homopolymers of N-vinylformamide.

Particularly preferred nonionic polymers are acrylic acid ester copolymers, homopolymers of vinylpyrrolidone and copolymers, and also polyvinylcaprolactam.

Very particularly preferred nonionic polymers are homopolymers of vinylpyrrolidone e.g. Luviskol® K from BASF, copolymers of vinylpyrrolidone and vinyl acetate e.g. Luviskol® VA grades from BASF or PVPVA® S630L from ISP, terpolymers of vinylpyrrolidone, vinyl acetate and propionate such as e.g. Luviskol® VAP from BASF and polyvinylcaprolactams e.g. Luviskol® PLUS from BASF.

Advantageous anionic polymers are homo- or copolymers with monomer units containing acid groups, which are optionally copolymerized with comonomers which contain no acid groups. Suitable monomers are unsaturated, free-radically polymerizable compounds which have at least one acid group, and in particular carboxylic acid, sulfonic acid or phosphonic acid.

Examples of anionic polymers which contain carboxylic acid groups are:
acrylic acid or methacrylic acid homo- or copolymers or salts thereof. These include for example the copolymers of acrylic acid and acrylamides and/or sodium salts thereof, copolymers of acrylic acid and/or methacrylic acid and an unsaturated monomer selected from the group ethylene, styrene, vinyl ester, acrylic ester, methacrylic ester, optionally ethoxylated compounds, copolymers of vinylpyrrolidones, acrylic acid and C1-C20 alkyl methacrylates, e.g. Acrylidone® LM from ISP, copolymers of methacrylic acid, ethyl acrylates and tert-butyl acrylates e.g. Luvimer® 100 P from BASF;
crotonic acid derivative homo- or copolymers or salts thereof. These include for example vinyl acetate/crotonic acid, vinyl acetate/acrylate and/or vinyl acetate/vinyl neodecanoate/crotonic acid copolymers e.g. Resyn® 28-1310 or Resyn® 28-2930 from AkzoNobel or Luviset® CAN from BASF, sodium acrylate/vinyl alcohol copolymers;
unsaturated C4-C8 carboxylic acid derivatives or carboxylic anhydride copolymers selected from copolymers of maleic acid or maleic anhydride or fumaric acid or fumaric anhydride or itaconic acid or itaconic anhydride and at least one monomer selected from the group vinyl ester, vinyl ether, vinyl halogen derivatives, phenyl vinyl derivatives, acrylic acid, acrylic esters or copolymers of maleic acid or maleic anhydride or fumaric acid or fumaric anhydride or itaconic acid or itaconic anhydride and at least one monomer selected from the group of allyl esters, methallyl esters and optionally acrylamides, methacrylamides, alpha-olefin, acrylic esters, methacrylic esters, vinylpyrrolidones. Further preferred polymers are methyl vinyl ether/maleic acid copolymers which are formed by hydrolysis of vinyl ether/maleic anhydride copolymers. These polymers can also be partially esterified (ethyl, isopropyl or butyl esters) or partially amidated;
water-soluble or -dispersible anionic polyurethanes, e.g. Luviset® PUR from BASF, which are different from the polyurethanes according to the invention.

Advantageous anionic polymers comprising sulfonic acid groups are salts of polyvinyl sulfonic acids, polystyrene sulfonic acids such as e.g. sodium polystyrene sulfonate or of polyacrylamide sulfonic acids.

Particularly advantageous anionic polymers are acrylic acid copolymers, crotonic acid derivative copolymer, copolymers of maleic acid or maleic anhydride or fumaric acid or fumaric anhydride or itaconic acid or itaconic anhydride and at least one monomer selected from the group vinyl esters, vinyl ethers, vinyl halogen derivatives, phenyl vinyl derivatives, acrylic acid, acrylic esters and salts of polystyrene sulfonic acids.

Very particularly advantageous anionic polymers are acrylate copolymers, e.g. Luvimer® from BASF, ethyl acrylate/N-tert-butylacrylamide/acrylic acid copolymers ULTRAHOLD® STRONG from BASF, VA/crotonate/vinyl neodecanoate copolymer e.g. Resyn® 28-2930 from AkzoNobel, copolymers such as copolymers of methyl vinyl ether and maleic anhydride partially esterified e.g. GANTREZ® from ISP and sodium polystyrene sulfonates e.g. Flexan® 130 from AkzoNobel.

Advantageous amphoteric polymers can be selected from among the polymers which contain units A and B distributed randomly in the polymer chain, where A is a unit derived from a monomer with at least one basic nitrogen atom, and B is a unit derived from an acidic monomer which has one or more carboxy or sulfonic acid groups. Alternatively, A and B can be groups which are derived from zwitterionic carboxybetaine monomers or sulfobetaine monomers. A and B can also be a cationic polymer chain which comprise primary, secondary, tertiary or quaternary groups, where at least one amino group carries a carboxy group or sulfonic acid group which is bonded via a hydrocarbon group, or A and B are part of a polymer chain with ethylene-α,β-dicarboxylic acid unit in which the carboxylic acid groups have been reacted with a polyamine which contains one or more primary or secondary amino groups.

Particularly advantageous amphoteric polymers are:
polymers which are formed during the copolymerization of a monomer derived from a vinyl compound with carboxy group, such as in particular acrylic acid, methacrylic acid, maleic acid, α-chloroacrylic acid, and a basic monomer. The basic monomer is derived from a vinyl compound which is substituted and contains at least one basic atom, such as in particular dialkylaminoalkyl methacrylate and acrylate, dialkylaminoalkyl methacrylamide and -acrylamide. Such compounds have been described in the American patent U.S. Pat. No. 3,836,537.
polymers with units which are derived from: a) at least one monomer which is selected from among the acrylamides or methacrylamides which are substituted on the nitrogen atom with an alkyl group, b) at least one acidic comonomer which contains one or more reactive carboxy groups, and c) at least one basic comonomer, such as esters of acrylic acid and methacrylic acid with primary, secondary, tertiary and quaternary amino substituents and the quaternization product of dimethylaminoethyl methacrylate with dimethyl sulfate or diethyl sulfate.
Particularly preferred N-substituted acrylamides or methacrylamides are compounds whose alkyl groups contain 2 to 12 carbon atoms. Very particular preference is given to N-ethyl acrylamide, N-t-butyl acrylamide, N-t-octyl acrylamide, N-octyl acrylamide, N-decyl acrylamide, N-dodecyl acrylamide, and the corresponding methacrylamides.
Suitable acidic comonomers are selected in particular from the group acrylic acid, methacrylic acid, crotonic acid, itaconic acid, maleic acid, fumaric acid, and the alkyl monoesters having 1 to 4 carbon atoms of maleic acid, maleic anhydride, fumaric acid or fumaric anhydride.
Preferred basic comonomers are aminoethyl methacrylate, butylaminoethyl methacrylate, N,N-dimethylaminoethyl methacrylate, N-t-butylaminoethyl methacrylate.
Crosslinked and completely or partially acylated polyaminoamides which are derived from polyaminoamides of the following general formula

—[CO—R—CO—Z]—
in which R is a divalent group which is derived from a saturated dicarboxylic acid, an aliphatic mono- or dicarboxylic acid with ethylenic double bond, an ester of these acids with a lower alkanol having 1 to 6 carbon atoms or a group which is formed upon the addition of one of these acids onto a bis primary or bis secondary amine, and Z is a group which is derived from a bis-primary, mono- or bis-secondary polyalkylene polyamine.
The saturated carboxylic acids are preferably selected from among the acids having 6 to 10 carbon atoms, such as adipic acid, 2,2,4-trimethyl adipic acid and 2,4,4-trimethyl adipic acid, terephthalic acid; acids with an ethylenic double bond, such as, for example, acrylic acid, methacrylic acid and itaconic acid.
The alkane sultones used for the acylation are preferably propanesultone or butanesultone, the salts of the acylating agents are preferably the sodium salts or potassium salts.
Polymers with zwitterionic units of the following formula:

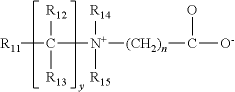

in which R₁₁ is a polymerizable unsaturated group, such as acrylate, methacrylate, acrylamide or methacrylamide, y and z are integers from 1 to 3, R₁₂ and R₁₃ are a hydrogen atom, methyl, ethyl or propyl, R₁₄ and R₁₅ are a hydrogen atom or an alkyl group which is selected such that the sum of the carbon atoms R₁₄ and R₁₅ does not exceed 10.

Polymers which contain such units can also have units which stem from non-zwitterionic monomers, such as dimethyl- and diethylaminoethyl acrylate or dimethyl- and diethylaminoethyl methacrylate or alkyl acrylates or alkyl methacrylates, acrylamides or methacrylamides or vinyl acetate.
Polymers which are derived from chitosan and contain monomer units which correspond to the following formulae:

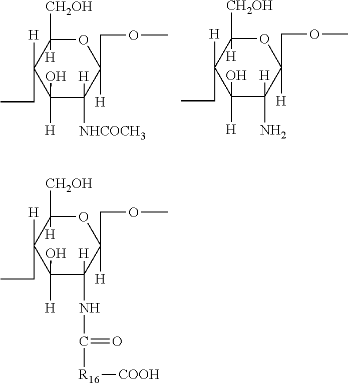

where the first unit is present in quantitative fractions of 0 to 30%, the second unit is present in quantitative fractions of 5 to 50% and the third unit is present in quantitative fractions of 30 to 90%, with the proviso that in the third unit R₁₆ is a group of the following formula:

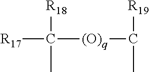

in which if q=0, the groups R₁₇, R₁₈ and R₁₉ are identical or different and are in each case a hydrogen atom, methyl, hydroxy, acetoxy or amino, a monoalkylamine radical or a dialkylamine radical, which optionally interrupted by one or more nitrogen atoms and/or optionally with one or more groups amino, hydroxy, carboxy, alkylthio, sulfonic acid, alkylthio, the alkyl group of which carries an amino radical, where at least one of the groups R₁₇, R₁₈ and R₁₉ is in this case a hydrogen atom; or if q=1, the groups R₁₇, R₁₈ and R₁₉ are in each case a hydrogen atom, and also the salts which form these compounds with bases or acids.
Polymers which correspond to the following general formula and which are described for example in the French patent 1 400 366:

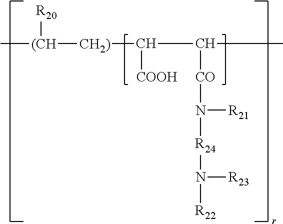

in which R₂₀ is a hydrogen atom, CH₃O, CH₃CH₂O or phenyl, R₂₁ is a hydrogen atom or a lower alkyl group, such as methyl or ethyl, R₂₂ is a hydrogen atom or a lower C₁₋₆-alkyl group, such as methyl or ethyl, R₂₃ is a lower C₁₋₆-alkyl group, such as methyl or ethyl or a group of the formula: —R₂₄—N(R₂₂)₂, where R₂₄ is a group —CH₂—CH₂, —CH₂—CH₂—CH₂— or —CH₂—CH(CH₃)— and where R₂₂ has the meanings given above.
Polymers which can be formed during the N-carboxyalkylation of chitosan, such as N-carboxymethylchitosan or N-carboxybutylchitosan.
Alkyl(C₁₋₅) vinyl ether/maleic anhydride copolymers which are partially modified partially by semiamidation with an N,N-dialkylaminoalkylamine such as N,N-dimethylaminopropylamine or an N,N-dialkylamino alcohol. These polymers can also contain further comonomers, such as vinylcaprolactam.

Very particular advantageous amphoteric polymers are e.g. the copolymers octylacrylamide/acrylates/butylaminoethyl methacrylate copolymer, which are commercially available under the names AMPHOMER®, AMPHOMER® LV 71 or BALANCE® 47 from AkzoNobel, and methyl methacrylate/methyldimethylcarboxymethylammonium ethyl methacrylate copolymers.

It is optionally advantageous to neutralize the anionic and amphoteric polymers with suitable bases to improve their solubility in water or their dispersibility in water.

Neutralizing agents for polymers which contain acid groups that can be used are the following bases: hydroxides whose cation is ammonium or an alkali metal, such as e.g. NaOH or KOH.

Other neutralizing agents are primary, secondary or tertiary amines, amino alcohols or ammonia. Preference is given here to using 2-amino-2-methyl-1,3-propanediol (AMPD), 2-amino-2-ethyl-1,3-propanediol (AEPD), 2-amino-2-methyl-1-propanol (AMP), 2-amino-1-butanol (AB), 2-amino-1,3-propanediol, monoethanolamine (MEA), diethanolamine (DEA), triethanolamine (TEA), monoisopropanolamine (MIPA), diisopropanolamine (DIPA), triisopropanolamine (TIPA), dimethyl laurylamine (DML), dimethyl myristalamine (DMM), and dimethyl stearamine (DMS).

The neutralization can take place partially or completely depending on the intended application.

It is optionally possible, but less preferred, to use cationic polymers, such as for example polymers which contain primary, secondary, tertiary and/or quaternary amino groups which are bonded part of the polymer chain or directly to the polymer chain.

The compositions according to the invention can furthermore contain thickeners. Advantageous thickeners are:
Crosslinked or non-crosslinked acrylic acid or methacrylic acid homo- or copolymers. These include crosslinked homopolymers of methacrylic acid or acrylic acid, copolymers of acrylic acid and/or methacrylic acid and monomers which are derived from other acrylic or vinyl monomers, such as C10-30 alkyl acrylates, C10-30-alkyl methacrylates and vinyl acetate.
Thickening polymers of natural origin, for example based on cellulose, guar gum, xanthan, scleroglucan, gellan gum, rhamsan and karaya gum, alginates, maltodextrin, starch and its derivatives, carob seed flour, hyaluronic acid.
Nonionic, anionic, cationic or amphoteric associative polymers e.g. based on polyethylene glycols and their derivatives, or polyurethanes.
Crosslinked or non-crosslinked homopolymers or copolymers based on acrylamide or methacrylamide, such as homopolymers of 2-acrylamido-2-methylpropanesulfonic acid, copolymers of acrylamide or methacrylamide and methacryloyloxyethyltrimethylammonium chloride or copolymers of acrylamide and 2-acrylamido-2-methylpropanesulfonic acid are given.

Particular advantageous thickeners are thickening polymers of natural origin, crosslinked acrylic acid or methacrylic acid homo- or copolymers and crosslinked copolymers of 2-acrylamido-2-methylpropanesulfonic acid.

Very particular advantageous thickeners are xanthan gum, such as the products supplied under the names Keltrol® and Kelza® from CP Kelco or the products from RHODIA with the name Rhodopol and guar gum, such as the products available under the name Jaguar® HP105 from RHODIA.

Very particular advantageous thickeners are crosslinked homopolymers of methacrylic acid or acrylic acid which are commercially available from Lubrizol under the names Carbopol® 940, Carbopol® 941, Carbopol® 980, Carbopol® 981, Carbopol® ETD 2001, Carbopol® EDT 2050, Carbopol® 2984, Carbopol® 5984 and Carbopol® Ultrez 10, from 3V under the names Synthalen® K, Synthalen® L and Synthalen® MS and from PROTEX under the names Modarez® V 1250 PX, Modarez® V2000 PX, Viscaron® A1600 PE and Viscaron® A700 PE.

Very particular advantageous thickeners are crosslinked copolymers of acrylic acid or methacrylic acid and a C₁₀₋₃₀-alkyl acrylate or C₁₀₋₃₀-alkyl methacrylate and copolymers of acrylic acid or methacrylic acid and vinylpyrrolidone. Such copolymers are commercially available for example from Lubrizol under the names Carbopol® 1342, Carbopol® 1382, Pemulen® TR1 or Pemulen® TR2 and from ISP under the names Ultrathix P-100 (INCI: Acrylic Acid/VP Crosspolymer).

Very particular advantageous thickeners are crosslinked copolymers of 2-acrylamido-2-methylpropanesulfonic acid. Such copolymers are available for example from Clairant under the names Aristoflex® AVC (INCI: Ammonium Acryloyldimethyltaurate/VP Copolymer).

If the thickeners are used, they are generally present in a concentration of 0% to 2% by weight, preferably 0% to 1% by weight.

The compositions according to the invention can furthermore comprise a propellant gas.

Preferred propellant gases are hydrocarbons such as propane, isobutane and n-butane, and mixtures thereof. Compressed air, carbon dioxide, nitrogen, nitrogen dioxide and dimethyl ether, as well as mixtures of all of these gases can likewise be used.

The person skilled in the art is naturally aware that there are propellant gases that are nontoxic per se which would in principle be suitable for realizing the present invention in the form of aerosol preparations but which nevertheless should be dispensed with on account of an unacceptable impact on the environment or other accompanying phenomena. These are in particular fluorocarbons and chlorofluorocarbons (CFCs) such as e.g. 1,2-difluoroethane (propellant 152 A).

Furthermore, hair care active ingredients may be present in the compositions according to the invention. Care substances which can preferably be used are cyclic polydimethylsiloxanes (cyclomethicones) or silicone surfactants (polyether-modified siloxanes) of the dimethicone copolyol type or simethicone type. Cyclomethicones are supplied inter alia under the trade names Abil® K4 by Goldschmidt or e.g. DC 244, DC 245 or DC 345 by Dow Corning. Dimethicone copolyols are supplied e.g. under the trade name DC 193 by Dow Corning or Belsil® DM 6031 by Wacker.

Optionally, conventional additives can likewise be present in the composition, for example in order to impart certain modifying properties to it. These may be for example silicones or silicone derivatives, wetting agents, humectants, softeners such as glycerol, glycol and phthalic esters and ethers, fragrances and perfumes, UV absorbers, dyes, pigments, and other colorants, anticorrosive agents, neutralizing agents, antioxidants, anti-sticking agents, combining agents and conditioning agents, antistatic agents, shine agents, preservatives, proteins and derivatives thereof, amino acids, vitamins, emulsifiers, surface-active agents, viscosity modifiers, thickeners and rheology modifiers, gelling agents, opacifiers, stabilizers, surfactants, sequestrants, complexing agents, pearlizing agents, esthetic boosters, fatty acids, fatty alcohols, triglycerides, botanical extracts, clarifying auxiliaries and film formers.

These additives are generally present in a concentration of about 0.001% to 15% by weight, preferably 0.01% to 10% by weight, based on the total weight of the composition.

The compositions according to the invention can advantageously be present in a pump spray or aerosol packaging. They can also advantageously be foamed using a propellant gas. Correspondingly, pump sprays, aerosol packagings and foam dispensers which contain the composition according to the invention are likewise part of the invention.

A further preferred embodiment of the composition according to the invention is in the form of a spray which additionally comprises one or more of the following constituents: cosmetically suitable solvents, such as aliphatic alcohols with 2-4 carbon atoms, preferably ethanol, polyols, acetone, unbranched or branched hydrocarbons, cyclic hydrocarbons and mixtures thereof, and also propellant gases such as hydrocarbons, compressed air, carbon dioxide, nitrogen, nitrogen dioxide, dimethyl ether, fluorocarbons and chlorofluorocarbons, preferably dimethyl ether and/or a propane/butane mixture.

The present invention is illustrated by reference to the following examples. Unless stated otherwise, all quantitative data, fractions and percentages are based on the weight and the total amount or on the total weight of the composition.

### EXAMPLES

Unless indicated otherwise, all percentages refer to the weight.

Unless noted otherwise, all analytical measurements refer to measurements at temperatures of 23° C.

The solid contents or solid-body contents were ascertained in accordance with DIN EN ISO 3251 by heating a weighed sample at 105° C. to constant weight. At constant weight, the solid-body content was calculated by reweighing the sample.

Unless expressly mentioned otherwise, NCO values were determined volumetrically in accordance with DIN-EN ISO 11909.

The monitoring of free NCO groups was carried out by means of IR spectroscopy (band at 2260 cm⁻¹).

The stated viscosities were determined by means of rotary viscometry in accordance with DIN 53019 at 23° C. using a rotary viscometer from Anton Paar Germany GmbH, Ostfildern, Germany.

Determination of the average particle sizes (the number average is stated) of the polyurethane dispersions was carried out after dilution with deionized water by means of laser correlation spectroscopy (instrument: Malvern Zetasizer 1000, Malver Inst. Limited).

Substances Used and Abbreviations:

Diaminosulfonate: NH₂—CH₂CH₂—NH—CH₂CH₂—SO₃Na (45% strength in water)

### EXAMPLE ACCORDING TO THE INVENTION

### Polyurethane Urea Dispersion A

1360.0 g of a polyester of adipic acid, hexanediol and neopentyl glycol with an average molecular weight of 1700 g/mol were heated to 65° C. Then, 318.5 g of isophorone diisocyanate (IPDI) were added and the mixture was stirred at 105° C. until the NCO value was below the theoretical value. The finished prepolymer was dissolved with 3000 g of acetone at 50° C. and then a solution of 23.4 g of isophoronediamine (IPDA), 129.6 g of diaminosulfonate and 357 g of water was metered in. The after-stirring time was 15 min. The mixture was then dispersed by adding 2900 g of water. The solvent was removed by distillation in vacuo and a storage-stable dispersion was obtained, the solids content being adjusted by adding water.

| | |
|---|---|
| Solids content: | 32% |
| Particle size (LCS): | 27 nm |
| Viscosity: | 1500 mPas |
| pH: | 7.3 |

### EXAMPLE ACCORDING TO THE INVENTION

### Polyurethane Urea Dispersion B

318.8 g of a polyester of adipic acid, hexanediol and neopentyl glycol with an average molecular weight of 1700 g/mol were heated to 65° C. Then, 70.9 g of isophorone diisocyanate were added and the mixture was stirred at 105° C. until the NCO value was below the theoretical value. The finished prepolymer was dissolved with 700 g of acetone at 50° C. and then a solution of 3.5 g of isophoronediamine, 30.4 g of diaminosulfonate and 84 g of water was metered in. The after-stirring time was 15 min. The mixture was then dispersed by adding 513 g of water. The solvent was removed by distillation in vacuo and a storage-stable dispersion was obtained, the solids content being adjusted by adding water.

| | |
|---|---|
| Solids content: | 30% |
| Particle size (LCS): | 32 nm |
| Viscosity: | 1000 mPas |
| pH: | 7.2 |

### EXAMPLE ACCORDING TO THE INVENTION

### Polyurethane Urea Dispersion C

319 g of a polyester of adipic acid, hexanediol and neopentyl glycol with an average molecular weight of 1700 g/mol were heated to 65° C. Then, 79.2 g of isophorone diisocyanate were added and the mixture was stirred at 105° C. until the NCO value was below the theoretical value. The finished prepolymer was dissolved with 1100 g of acetone at 50° C. and then a solution of 8.0 g of isophoronediamine, 30.4 g of diaminosulfonate and 84 g of water was metered in. The after-stirring time was 15 min. The mixture was then dispersed by adding 540 g of water. The solvent was removed by distillation in vacuo and a storage-stable dispersion was obtained, the solids content being adjusted by adding water.

| | |
|---|---|
| Solids content: | 28% |
| Particle size (LCS): | 35 nm |
| Viscosity: | 760 mPas |
| pH: | 7.7 |

### COMPARATIVE EXAMPLE

### Polyurethane Urea Dispersion D

319 g of a polyester of adipic acid, hexanediol and neopentyl glycol with an average molecular weight of 1700 g/mol were heated to 65° C. Then, 56.0 g of isophorone diisocyanate and 14.1 g of hexamethylene diisocyanate were added and the mixture was stirred at 105° C. until the NCO value was below the theoretical value. The finished prepolymer was dissolved with 690 g of acetone at 50° C. and then a solution of 5.5 g of isophoronediamine, 30.4 g of diaminosulfonate and 84 g of water was metered in. The after-stirring time was 15 min. The mixture was then dispersed by adding 520 g of water. The solvent was removed by distillation in vacuo and a storage-stable dispersion was obtained, the solids content being adjusted by adding water.

| | |
|---|---|
| Solids content: | 31% |
| Particle size (LCS): | 71 nm |
| Viscosity: | 210 mPas |
| pH: | 7.4 |

### COMPARATIVE EXAMPLE

### Polyurethane Urea Dispersion E

319 g of a polyester of adipic acid, hexanediol and neopentyl glycol with an average molecular weight of 1700 g/mol were heated to 65° C. Then, 74.6 g of isophorone diisocyanate were added and the mixture was stirred at 105° C. until the NCO value was below the theoretical value. The finished prepolymer was dissolved with 700 g of acetone at 50° C. and then a solution of 4.1 g of isophoronediamine, 30.4 g of diaminosulfonate, 0.5 g of ethylenediamine and 87 g of water was metered in. The after-stirring time was 15 min. The mixture was then dispersed by adding 520 g of water. The solvent was removed by distillation in vacuo and a storage-stable dispersion was obtained, the solids content being adjusted by adding water.

| | |
|---|---|
| Solids content: | 30% |
| Particle size (LCS): | 47 nm |
| Viscosity: | 115 mPas |
| pH: | 7.6 |

### COMPARATIVE EXAMPLE

### Polyurethane Urea Dispersion F

319 g of a polyester of adipic acid, hexanediol and neopentyl glycol with an average molecular weight of 1700 g/mol were heated to 65° C. Then, 74.6 g of isophorone diisocyanate were added and the mixture was stirred at 105° C. until the NCO value was below the theoretical value. The finished prepolymer was dissolved with 700 g of acetone at 50° C. and then a solution of 3.7 g of isophoronediamine, 30.4 g of diaminosulfonate, 2.2 g of methylenebis(4-aminocyclohexane) and 100 g of water was metered in. The after-stirring time was 15 min. The mixture was then dispersed by adding 510 g of water. The solvent was removed by distillation in vacuo and a storage-stable dispersion was obtained, the solids content being adjusted by adding water.

| | |
|---|---|
| Solids content: | 30% |
| Particle size (LCS): | 38 nm |
| Viscosity: | 70 mPas |
| pH: | 6.7 |

### COMPARATIVE EXAMPLE

### Polyurethane Urea Dispersion G

319 g of a polyester of adipic acid, hexanediol and neopentyl glycol with an average molecular weight of 1700 g/mol were heated to 65° C. Then, 74.6 g of isophorone diisocyanate were added and the mixture was stirred at 105° C. until the NCO value was below the theoretical value. The finished prepolymer was dissolved with 700 g of acetone at 50° C. and then a solution of 30.4 g of diaminosulfonate, 6.8 g of methylenebis(4-aminocyclohexane) and 100 g of water was metered in. The after-stirring time was 15 min. The mixture was then dispersed by adding 510 g of water. The solvent was removed by distillation in vacuo and a storage-stable dispersion was obtained, the solids content being adjusted by adding water.

| | |
|---|---|
| Solids content: | 31% |
| Particle size (LCS): | 42 nm |
| Viscosity: | 70 mPas |
| pH: | 6.7 |

### COMPARATIVE EXAMPLE

### Polyurethane Urea Dispersion H

319 g of a polyester of adipic acid, hexanediol and neopentyl glycol with an average molecular weight of 1700 g/mol were heated to 65° C. Then, 88.1 g of methylenebis(4-cyclochexyl isocyanate) were added and the mixture was stirred at 105° C. until the NCO value was below the theoretical value. The finished prepolymer was dissolved with 730 g of acetone at 50° C. and then a solution of 5.5 g of isophoronediamine, 30.4 g of diaminosulfonate and 84 g of water was metered in. The after-stirring time was 15 min. The mixture was then dispersed by adding 540 g of water. The solvent was removed by distillation in vacuo and a storage-stable dispersion was obtained, the solids content being adjusted by adding water.

| | |
|---|---|
| Solids content: | 40% |
| Particle size (LCS): | 300 nm |
| Viscosity: | <50 mPas |
| pH: | 8.8 |

### COMPARATIVE EXAMPLE

### Polyurethane Urea Dispersion I

340 g of a polyester of adipic acid, hexanediol and neopentyl glycol with an average molecular weight of 1700 g/mol were heated to 65° C. Then, 60.1 g of hexamethylene diisocyanate were added and the mixture was stirred at 105° C. until the NCO value was below the theoretical value. The finished prepolymer was dissolved with 711 g of acetone at 50° C. and then a solution of 2.1 g of ethylenediamine, 32.4 g of diaminosulfonate and 104.3 g of water was metered in. The after-stirring time was 15 min. The mixture was then dispersed by adding 1880 g of water. The solvent was removed by distillation in vacuo and a storage-stable dispersion was obtained, the solids content being adjusted by adding water.

| | |
|---|---|
| Solids content: | 40% |
| Particle size (LCS): | 198 nm |
| Viscosity: | 700 mPas |
| pH: | 6.31 |

Application-Related Comparative Experiments:

Turbidity Measurement:

The optical properties of the polyurethane ureas according to the invention were investigated in three different mixtures:
a) VOC 80 mixtures: 2% by weight of the respective polyurethane urea (based on solids)+80% ethanol (denatured with 0.448% diethyl phthalate)+water made up to 100%
b) VOC 55 mixtures: 2% by weight of the respective polyurethane urea (based on solids)+55% ethanol (denatured with 0.448% diethyl phthalate)+water made up to 100%
c) VOC 0 mixtures: 2% by weight of the respective polyurethane urea (based on solids)+water made up to 100%

The turbidity of the respective mixtures was measured 1 day after preparation using a turbidity meter model 2100 P from Hach (turbidity measurement from 0.01 to 1000 NTU). Before the measurement, the instrument was calibrated with formazine standards (standard 1/20 NTU, standard 2/100 NTU and standard 3/800 NTU).

The results of the turbidity measurements are summarized in table 1 below:

TABLE 1

| | VOC 80 | VOC 55 | VOC 0 |
|---|---|---|---|
| Polyurethane urea | Turbidity values in _NTU | | |
| A | 13 | 39 | 82 |
| B | 74 | 40 | 109 |
| C | 43 | 46 | 119 |

The results of the turbidity measurements of mixtures of the polyurethane ureas of the comparative examples are shown in table 2.

| | | |
|---|---|---|
| VOC 80 | VOC 55 | VOC 0 |

| | |
|---|---|
| Polyurethane urea | Turbidity values in NTU |

| | | | |
|---|---|---|---|
| D | 80 | 102 | 282 |
| E | 134 | 60 | 147 |
| F | 105 | 65 | 115 |
| G | 121 | 63 | 173 |
| H | n.m (>1000) | n.m (>1000) | n.m (>1000) |

The experiments clearly show that mixtures with particularly low turbidity values are obtainable from the polyurethane ureas according to the invention; i.e. they are particularly well suited for producing transparent mixtures.

Curl Retention:

For the so-called “curl retention” experiments, commercially available European mixed hair (useful length: 16 cm, Kerling, weight: 0.7 g) was used. The hair was subjected to a standardized washing procedure prior to use. For this, the hair was softened in water for 15 minutes and then shampooed for one minute with 0.2 ml of standard shampoo, thoroughly rinsed with warm water, blow-dried on cold and conditioned at 21±1° C. and 50±5% relative humidity. 0.1 g of a 2% by weight polymer mixture was applied to the tress. Tresses of the hair prepared in this way were wound onto 16 mm rollers and then conditioned at 21±1° C. and 50±5% relative humidity for at least 18 hours.

The “curl retention” experiments were carried out in a special climatically controlled chamber at a relative humidity of 90±5%. The temperature in the chamber was 21±1° C. The prepared tresses were suspended simultaneously in the chamber. The length of the tresses was read off on a scale at different times. Each experiment was carried out with eight tresses.

The “curl retention” was calculated according to the following formula:

CR=(l_{f}−lₜᵢ)/(l_{f}−lₜ)×100;

in which l_{f} was the tress length, lₜᵢ was the original length of the curled hair tress and lₜ was the length of the curled hair tress at time t-.

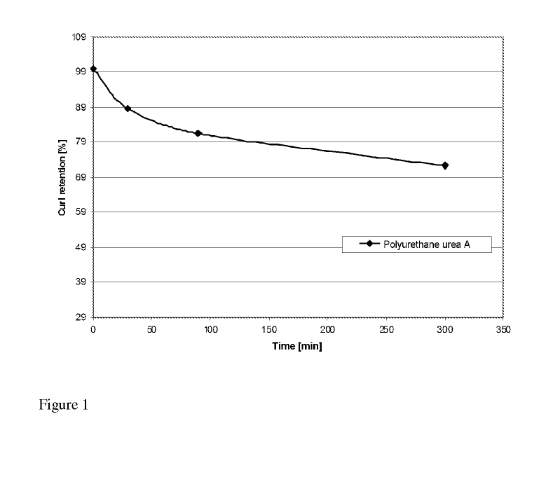

The results of the measurement are shown in graph form in FIG. 1. This clearly shows that a good curl retention is achieved with the polyurethane urea used according to the invention.

### APPLICATION EXAMPLES

Examples are given below of cosmetic formulations in which the compositions according to the invention are used. In each case it is stated how many parts by weight of the individual components are present in a formulation.

“Pump-Setting Spray”

| | A | B |
|---|---|---|
| Polyurethane urea according to | 2 | 10 |
| the invention (based on solids) | | |
| Ethanol | 90 | 55 |
| Perfume | q.s. | q.s. |
| Water | ad 100% | ad 100% |

q.s. quantum satis

Aerosol Hairsprays

| | C | D | E | F | G |
|---|---|---|---|---|---|
| Polyurethane urea according | 5 | 10 | 2 | 5 | 8 |
| to the invention (based on | | | | | |
| solids) | | | | | |
| Octylacrylamide/acrylate/ | | | | 1.8 | |
| butylaminoethyl methacrylate¹ | | | | | |
| (based on solids) | | | | | |
| Acrylate copolymer² | | | 2 | | |
| Aminomethylpropanol | | | q.s | q.s | |
| Glycerol | | 0.5 | | | |
| Panthenol | | | 0.5 | | 0.5 |
| PEG/PPG-18/18 dimethicone | | | | 0.5 | |
| PEG-12 dimethicone | | 0.05 | | | |
| Propylene glycol | | | | 0.5 | |
| Cyclomethicone | | | 1.0 | | 1.0 |
| Benzophenone-3 | | 0.1 | 0.1 | | 0.1 |
| Perfume | q.s | q.s | q.s | q.s | q.s |
| Ethanol | 30 | 25 | 70 | 50 | 50 |
| Water | ad | ad | ad | ad | ad |
| | 100% | 100% | 100% | 100% | 100% |
| Propane/butane 3.5 bar (20° C.) | | | 20 | 10 | |
| Dimethyl ether | 40 | 30 | | 30 | |
| Fluorocarbon 152 A | | | | | 20 |

¹Amphomer, AkzoNobel
²Luvimer P-100, BASF

Hair Mousse

| | H | I |
|---|---|---|
| Polyurethane urea according to the | 2 | 4 |
| invention (based on solids) | | |
| Glycerol | 0.1 | |
| Panthenol | 0.05 | 0.5 |
| Polyquaternium-164 | 2 | |
| Cetyltrimethylammonium chloride | 0.2 | 0.5 |
| PEG-12 dimethicone | | 0.5 |
| Cyclomethicone | | 0.5 |
| Benzophenone-3 | 0.1 | |
| Perfume | q.s. | q.s. |
| Ethanol | 5 | 10 |
| Water | ad 100% | ad 100% |
| Preservative | q.s. | q.s. |
| Propane/butane/dimethyl ether | 10 | 7 |
| Dimethyl ether | | 7 |
| Fluorocarbon 152 A | | 3 |

Hair Gel/Cream

| | J | K | L |
|---|---|---|---|
| Polyurethane urea according to the | 5 | 2 | 8 |
| invention (based on solids) | | | |
| Carbomer | 0.8 | | |
| Acrylic acid/VP Crosspolymer | | 0.5 | |
| Ammonium | | | 0.8 |
| acryloyldimethyltaurate/VP | | | |
| copolymer | | | |
| Glycerol | 0.5 | | |
| Panthenol | | 0.5 | 0.5 |
| Propylene glycol | | | 0.2 |
| Cyclomethicone | | 0.2 | |
| Neutralizing agent | q.s. | q.s. | q.s. |
| Perfume | q.s. | q.s. | q.s. |
| Ethanol | 6 | | |
| Water | ad 100% | ad 100% | ad 100% |
| Preservative | q.s. | q.s. | q.s. |

1. A polyurethane urea obtainable by reacting
a) a single polyisocyanate component,
b) at least one polymeric polyol component,
c) at least one hydrophilizing component and
d) a single amino-functional chain extender component,
where the polyisocyanate component a) comprises more than 75 mol % isophorone diisocyanate and the amino-functional chain extender component (d) comprises more than 75 mol % isophoronediamine

2. The polyurethane urea as claimed in claim 1, wherein said polyisocyanate component a) comprises ≧80 mol %, optionally ≧85 mol %, further optionally 95 or 100 mol %, isophorone diisocyanate.

3. The polyurethane urea as claimed in claim 1, wherein said polymeric polyol component b) has a number-average molecular weights of ≧400 and ≦8000 g/mol and/or an OH functionality of from 1.5 to 6.

4. The polyurethane urea as claimed in claim 1, wherein said polymeric polyol component b) comprises a polyester, optionally a polyester based on adipic acid, or said polymeric polyol component b) consists of a polyester.

5. The polyurethane urea as claimed in claim 1, wherein the hydrophilizing component c) is an anionically hydrophilizing component and optionally a sulfonate.

6. The polyurethane urea as claimed in claim 5, wherein the amino-functional chain extender component d) comprises ≧85 mol %, optionally ≧95 or 100 mol % isophoronediamine

7. A polyurethane urea as claimed in claim 1, capable of being used in cosmetics, optionally in a field of hair cosmetics, optionally in a field of hair styling.

8. A cosmetic composition comprising a solvent mixture and a polyurethane urea as claimed in claim 1, where the solvent mixture comprises ethanol and water.

9. The composition as claimed in claim 8, wherein the solvent mixture comprises ≧10% by weight and ≦98% by weight, optionally ≧20% by weight and ≦98% by weight, further preferably optionally ≧20% by weight and ≦90% by weight and/or optionally ≧20% by weight and ≦80% by weight, ethanol.

10. The composition as claimed in claim 8, wherein said solvent mixture consists of water and ethanol.

11. The composition as claimed in claim 8, wherein said composition comprises ≧0.1% by weight and ≦30% by weight, optionally ≧0.1% by weight and ≦20% by weight, further optionally ≧0.5% by weight and ≦15% by weight and/or optionally ≧0.5% by weight and ≦10% by weight, of the polyurethane urea.

12. The composition as claimed in claim 8, wherein said composition comprises ≧10% by weight and ≦98% by weight, optionally ≧20% by weight and ≦98% by weight, further optionally ≧30% by weight and ≦98% by weight and/or ≧40% by weight and ≦98% by weight, of the solvent mixture.

13. A cosmetic composition as claimed in claim 8 capable of being used in cosmetics, optionally in a field of hair cosmetics, optionally in a field of hairstyling.

14. A method for forming a hairstyle, comprising applying a cosmetic composition as claimed in claim 8 to hair.
